(19) United States
(12) Patent Application Publication
(10) Pub. No.: US 2006/0160853 A1
(43) Pub. Date: Jul. 20, 2006

(54) 2-[(4-Benzyl)-1-piperidinyl)methyl]benzimidazole-5-ol derivatives as nr2b receptor antagonists

(76) Inventors: John A McCauley, Maple Glen, PA (US); David A Claremon, Maple Glen, PA (US); Nigel J Liverton, Harleysville, PA (US)

Correspondence Address: MERCK AND CO., INC, P O BOX 2000, RAHWAY, NJ, 07065-0907, US

(21) Appl. No.: 10/535,788

(22) PCT Filed: Nov. 18, 2003
(86) PCT No.: PCT/US03/36884

§ 371 Date: May 20, 2005

Related U.S. Application Data
(60) Provisional application No. 60/428,410, filed on Nov. 22, 2002.

(51) Int. Cl. A61K 31/454; C07D 403/02

(52) U.S. Cl. 514/322; 546/199

(57) ABSTRACT

2-[(4-Benzyl)-1-piperidinyl)-methyl]benzimidazole-5-ol derivatives are NMDA NR2B receptor antagonists useful in the treatment of pain and other NMDA mediated diseases.

### BACKGROUND OF THE INVENTION

Glutamate plays a key role in processes related to chronic pain and pain-associated neurotoxicity—primarily by acting through N-methyl-D-aspartate (“NMDA”) receptors. Thus, inhibition of such action—by employing ion channel antagonists, particularly NMDA antagonists—can be beneficial in the treatment and control of pain.

Known NMDA antagonists include ketamine, dextromethorphan, and 3-(2-carboxypiperazin-4-yl)-propyl-1-phosphonic acid (“CPP”). Although these compounds have been reported (J. D. Kristensen, et al., Pain, 51:249-253 (1992); K. Eide, et al., Pain, 61:221-228 (1995); D. J. Knox, et al., Anaesth. Intensive Care 23:620-622 (1995); and M. B. Max, et al., Clin. Neuropharmacol. 18:360-368 (1995)) to produce symptomatic relief in a number of neuropathies including postherpetic neuralgia, central pain from spinal cord injury, and phantom limb pain, widespread use of these compounds is precluded by their undesirable side effects. Such side effects at analgesic doses include psychotomimetic effects such as dizziness, headache, hallucinations, dysphoria, and disturbances of cognitive and motor function. Additionally, more severe hallucinations, sedation, and ataxia are produced at doses only marginally higher than analgesic doses. Thus, it would be desirable to provide novel NMDA antagonists that are absent of undesirable side effects or that produce fewer and/or milder side effects.

NMDA receptors are heteromultimeric assemblies of subunits, of which two major subunit families designated NR1 and NR2 have been cloned. Without being bound by theory, it is generally believed that the various functional NMDA receptors in the mammalian central nervous system (“CNS”) are formed by combinations of NR1 and NR2 subunits. The NR2 subunit family is in turn divided into four individual subunit types: NR2A, NR2B, NR2C, and NR2D. I. Ishii, et al., J. Biol. Chem., 268:2836-2843 (1993), A. Wenel, et al., Neural Report, 7:4548 (1995), and D. J. Laurie et al., Mol. Brain Res., 51:23-32 (1997) describe how the various resulting combinations produce a variety of NMDA receptors differing in physiological and pharmacological properties such as ion gating properties, magnesium sensitivity, pharmacological profile, as well as in anatomical distribution.

For example, while NR1 is found throughout the brain, NR2 subunits are differentially distributed. In particular, it is believed that the distribution map for NR2B lowers the probability of side effects while producing pain relief. For example, S. Boyce, et al., Neuropharmacology, 38:611-623(1999) describes the effect of selective NMDA NR2B antagonists on pain with reduced side-effects. Thus, it would be desirable to provide novel NMDA antagonists that target the NR2B subunit-containing receptors.

Phenol compounds as NMDA antagonists are described in U.S. Pat. Nos. 5,306,723 and 5,436,255, and in International Patent Publications WO91/17156, WO92/19502, WO93/02052, WO94/29571, WO95/28057, WO96/37226, and EP 04422506. Benzyl piperidine substituted with phenols or imidazoles are described in Z. -L. Zhou, et al., J. Medicinal Chemistry, 42:2993-3000(1999); T. F. Gregory, et al., Poster #94, 218^{th} National Meeting American Chemical Society, New Orleans, La., Aug. 22-26, 1999. Other NMDA NR2B selective compounds are described in European Patent Publication EP 787493 and Bnitish J. Pharmacol., 123:463(1998). However, there continues to be a need for novel NMDA antagonists that target the NR2B receptor.

International Patent Publication WO94/21615 describes dopamine D4 antagonist benzimidazole-piperidine compounds of the formula: 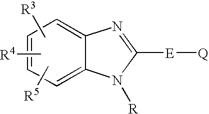
where Q may be a substituted piperidyl moiety. These compounds are disclosed in International Patent Publication WO01/30330 as being NMDA NR2B antagonists useful for the treatment of pain.

International Patent Publication WO01/32615 describes 1,4-disubstituted NMDA/NR2B antagonists having the formula: 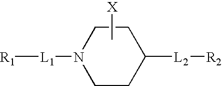
wherein R₁ and R₂ may be optionally substituted phenyl or benzimidazolyl group.

International Patent Publication WO02/34718 describes NR2B selective NMDA antagonists of the formula:

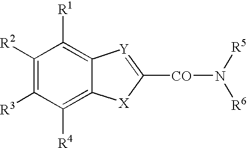

### SUMMARY OF THE INVENTION

The present invention relates to novel 2-[(4-benzyl)-1-piperidinyl)-methyl]benzimidazole-5-ol derivatives, pharmaceutical compositions utilizing the compounds, and novel methods to treat pain and Parkinson's Disease by utilizing the compounds.

### DETAILED DESCRIPTION OF THE INVENTION

The present invention provides compounds having the formula I: 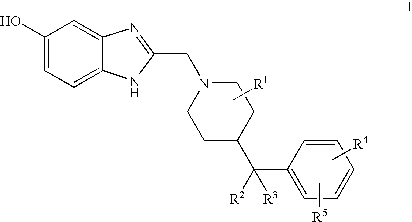
and pharmaceutically acceptable salts thereof, wherein
R¹ is H or OH;
R² is H or OH;
R³ is H, or R³ and R² together represent oxo;
R⁴ and R⁵ are independently H, halogen, C₁₋₆alkyl, C₁₋₆alkoxy or trifluoromethyl.

In one subset of formula I are compounds wherein R¹, R², and R³ are each H. In another subset of formula I are compounds wherein R¹ is OH.

Representative compounds of the present invention are provided in the following Table:

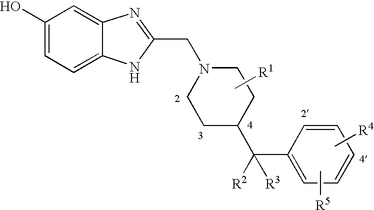

| R¹ | R² | R³ | R⁴ | R⁵ |
|---|---|---|---|---|
| H | H | H | H | H |
| H | H | H | H | 2′-F |
| H | H | H | H | 3′-F |
| H | H | H | H | 4′-F |
| H | H | H | 2′-F | 6′-F |
| H | H | H | H | 4-CH₃ |
| H | H | H | H | 4-CF₃ |
| 4-OH | H | H | H | H |
| H | oxo | | H | H |
| H | OH | H | H | H |
| 3-OH | H | H | H | H |
| 3-OH | H | H | H | 4-CH₃ |

In another aspect the present invention provides pharmaceutical compositions comprising a therapeutically effective amount of a compound of formula I and a pharmaceutically acceptable carrier.

In another aspect the present invention provides a method for the treatment of pain comprising administering to a patient in need thereof a therapeutically effective amount of a compound of formula I. In one subset the pain is neuropathic pain such as postherpetic neuralgia and diabetic neuropathy.

In yet another aspect the present invention provides a method for the treatment of Parkinson's disease comprising administering to a patient in need thereof a therapeutically effective amount of a compound of formula I.

Optical Isomers-Diastereomers-Geometric Isomers-Tautomers

Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The above Formula I is shown without a definitive stereochemistry at certain positions. The present invention includes all stereoisomers of Formula I and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general Formula I may be obtained by stereospecific synthesis using optically pure starting materials or reagents of known configuration.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula I.

Salts

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric, and tartaric acids.

Prodrugs

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu.

Pharmaceutical Compositions

The pharmaceutical compositions of the present invention comprise a compound represented by Formula I (or pharmaceutically acceptable salts thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 1 mg to about 500 mg of the active ingredient and each cachet or capsule preferably containing from about 1 to about 500 mg of the active ingredient.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

Utilities

Compounds of formula I are NMDA NR2B receptor antagonists, and as such are useful for the treatment and prophylaxis of diseases and disorders mediated through the NR2B receptor. Such diseases and disorders include, but are not limited to, neuropathic pain (such as postherpetic neuralgia, nerve injury, the “dynias”, e.g., vulvodynia, phantom limb pain, root avulsions, painful diabetic neuropathy, painful traumatic mononeuropathy, painful polyneuropathy), central pain syndromes (potentially caused by virtually any lesion at any level of the nervous system), and postsurgical pain syndromes (eg, postmastectomy syndrome, postthoracotomy syndrome, stump pain)), bone and joint pain (osteoarthritis), repetitive motion pain, dental pain, cancer pain, myofascial pain (muscular injury, fibromyalgia), perioperative pain (general surgery, gynecological), chronic pain, dysmennorhea, as well as pain associated with angina, and inflammatory pain of varied origins (e.g. osteoarthritis, rheumatoid arthritis, rheumatic disease, teno-synovitis and gout), migraine and cluster headache, depression, anxiety, schizophrenia, stroke, traumatic brain injury, cerebral ischemia, amyotrophic lateral sclerosis, Huntington's disease, Parkinson's disease, sensorineural hearing loss, tinnitis, neurological damage caused by epileptic seizures or by neurotoxin poisoning or by impairment of glucose and/or oxygen to the brain, vision loss caused by neurodegeneration of the visual pathway, Restless Leg Syndrome, multi-system atrophy, non-vascular headache, primary hyperalgesia, secondary hyperalgesia, primary allodynia, secondary allodynia, or other pain caused by central sensitization. Compounds of formula I may be used to prevent dyskinesias, particularly the side effects accompanying normal doses of L- Dopa. Furthermore, compounds of formula I may be used to decrease tolerance and/or dependence to opioid treatment of pain, and for treatment of withdrawal syndrome of e.g., alcohol, opioids, and cocaine.

Combination Therapy

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (1) non-steroidal anti-inflammatory agents; (2) COX-2 inhibitors; (3) bradykinin B1 receptor antagonists; (4) sodium channel antagonists; (5) nitric oxide synthase (NOS) inhibitors; (6) glycine site antagonists; (7) potassium channel openers; (8) AMPA/kainate receptor antagonists; (9) calcium channel antagonists; (10) GABA-A receptor modulators (e.g., a GABA-A receptor agonist); (11) matrix metalloprotease (MMP) inhibitors; (12) thrombolytic agents; (13) opioids such as morphine; and (14) neutrophil inhibitory factor (NIF).

Experimental Protocols for Biological Evaluation

(a) Assessing the Activity of Selected Compounds to Inhibit NR1A/2B NMDA Receptor Function (FLIPR Assay)

The activity of selected compounds to inhibit NR1A/2B NMDA receptor function measured as NR1A/2B receptor-mediated Ca²⁺ influx is assessed by the following procedure:

NR1A/2B receptor transfected L(tk) cells are plated in 96-well format at 3×10⁶ cells per plate and grown for one-two days in normal growth media (Dulbeccos MEM with Na pyruvate, 4500 mg glucose, pen/strep, glutamine, 10% FCS and 0.5 mg/ml geneticin). NR1A/2B-expression in these cells is induced by the addition of 4 nM dexamethasone in the presence of 500 μM ketamine for 16-24 hours. After receptor induction cells are washed using a Labsystem Cellwasher two times with assay buffer (Hanks balanced salt solution (HBSS-Mg⁺⁺ free) containing 20 mM HEPES, 0.1% BSA, 2 mM CaCl₂ and 250 μM probenecid). The cells in all wells are loaded with the Ca⁺⁺ sensitive dye Fluo-3 (Molecular Probes, Inc.) at 4 μM in assay buffer containing 0.5% FBS, and 0.04% Pluronic F-127 (Molecular Probes, Inc.) for 1 h at 37° C. avoiding light. The cells are then washed with the Cellwasher four times with assay buffer leaving them in 100 μl buffer. Test compounds in solution are pipetted by FLIPR (Fluorometric Imaging Plate Reader) into each test well for a 2 min pretreatment. During this time the fluorescence intensity is recorded (excitation at 488 nm and emission at 530 nm). The agonist solution (glutamate/glycine, 50 μL, final concentration 1 μM/1 μM) is then added by FLIPR into each well already containing 150 μL of buffer (containing the test compound or vehicle) and the fluorescence is continuously monitored for 10 min. The endpoint fluorescence values are used to determine an IC₅₀ value comparing the agonist-stimulated signal for the vehicle alone sample and that for the cells incubated with each concentration of test compound.

(b) Determining the Apparent Dissociation Constant (Ki) of Compounds for Human NR1A/NR2B Receptors (Binding Assay):

The radioligand binding assay is performed at room temperature in 96-well microtiter plates with a final assay volume of 1.0 mL in 20 mM Hepes buffer (pH 7.4) containing 150 mM NaCl. Solutions of test compounds were prepared in DMSO and serially diluted with DMSO to yield 20 μL of each of 10 solutions differing by 3-fold in concentration. Non-specific binding (NSB) was assessed using AMD-1 (10 μM final concentration) and total binding (TB) was assessed by using DMSO. A solution of NR1A/NR2B receptors (40 pM final concentration) and tritiated AMD-2 (1 nM final concentration) were added to the test compounds. After 3 h of incubation at room temperature, samples are filtered through Packard GF/B filters (presoaked in 0.05% PEI, polyethyleninine Sigma P-3143) and washed 10 times with 1 mL of cold 20 mM Hepes buffer per wash. After vacuum drying of the filter plates, 40 μL of Packard Microscint-20 was added and bound radioactivity determined in a Packard TopCount. The apparent dissociation constant (Ki), the maximum percentage inhibition (% Imax), the minimum percentage inhibition (% Imin) and the hill slope (nH) were determined by a non-linear least squares fitting the bound CPM data to Equation #1 below.

$$Equation \#1 \text{:} \\ CPM Bound = \frac{( SB ) ( \% {I}_{max} - \% {I}_{min} ) / 100}{( 1 + {( [ Drug ] / ( Ki [ AMD - 2 ] / {K}_{D} ) )}^{nH} )} + NSB + ( SB ) ( 100 - \% {I}_{max} ) / 100$$

where, K_{D} is the apparent dissociation constant for the radioligand for the receptor as determined by hot saturation and SB is the specifically bound CPM determined from the difference of TB and NSB.

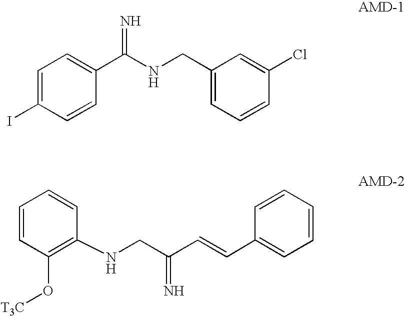

Compounds AMD-1 and AMD-2 can be synthesized in accordance with the following general reaction schemes.

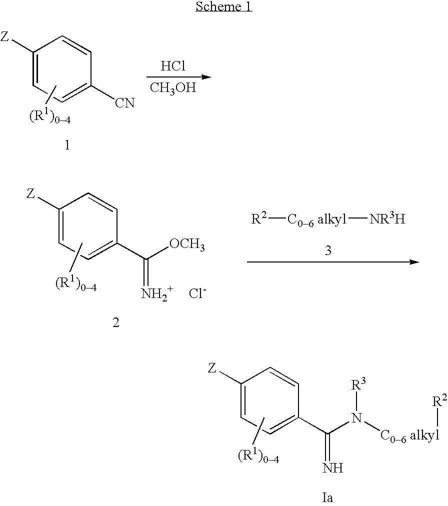

In accordance with Scheme 1, hydrogen chloride is bubbled through a solution of the appropriately substituted benzonitrile 1 in methanol at room temperature. The volatiles are removed under reduced pressure and the resulting residue is triturated with ether and filtered to yield the desired imidate 2. Imidate 2 is dissolved in methanol at ambient temperature, treated with amine 3 at ambient temperature and stirred under argon. The volatiles are removed under reduced pressure and the residue purified by preparative HPLC or trituration with ether to afford amidine Ia.

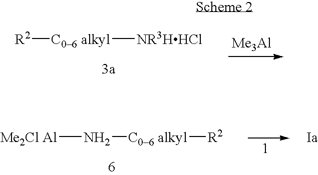

In accordance with Scheme 2, at rt under argon, amine 3a is dissolved in ether and was treated with 1 M hydrogen chloride in ether (1 equiv.) in a single portion. The resulting precipitate is stirred vigorously for 10 minutes. The volatiles are removed under reduced pressure. The residue is suspended in toluene, cooled to 0° C. under argon, treated with 2.0M trimethylaluminum (1.05 equiv.) in a dropwise manner, and stirred for 45 min at rt to afford intermediate 6 (not isolated). Compound 6 is added to a solution of nitrile 1 in toluene. The reaction is heated to 80° C. without stirring in a sealed tube for 18 h, cooled to ambient temperature, poured onto a silica gel column and eluted with methanol/dichloromethane to give the amidine Ia.

### Synthesis of Tritiated AMD-2

Tritiated AMD-2 was prepared by the following procedure: The phenol of AMD-2 (2 mg, 0.008 mmol) dissolved in dimethylformamide (0.6 mL) and potasium carbonate (1.2 mg) for 1 hr. High specific activity tritiated methyl iodide (50 mCi, 0.0006 mmol, in toluene 1 mL, American Radiolabeled Chemicals) was added at room temperature and stirred for 2 hours. The reaction mixture was filtered using a Whatman PTFE 0.45 μm syringeless filter device to remove any insoluable potassium carbonate, washed with Abs. ethanol (2 mL, Pharmco), and the combined filtrates were concentrated to dryness at room temperature using a rotary evaporator; this also removed any unreacted tritiated methyl iodide. The residue was purified by HPLC chromatography on a Phenomenx Luna C8 semi-prep column (Luna 5 micro C8(2), 250×10.0 mm) using a gradient system of 20/80 acetonitrile/water with 0.1% trifluoroacetic acid to 100% acetronitrile with 0.1% trifluoroacetic acid in 20 min. Total activity of the product was 8 mCi. Further purification was effected by absorption onto a Waters C-18 Sep-pak column (Waters Sep-Pak PLUS C18) and elution with water followed by absolute ethanol. The product was diluted with absolute ethanol (10 mL) before submission for final analysis.

Compounds of formula I can be prepared according to the procedure depicted in the following scheme:

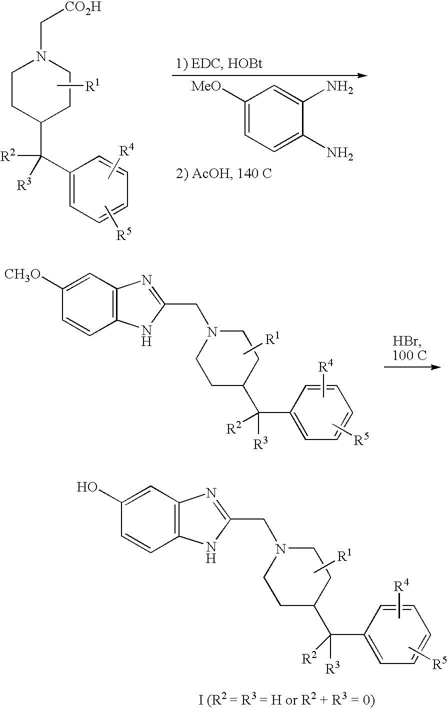

Thus, a 4-benzylpiperidin-1-acetic acid compound is reacted with 4-methoxy-1,2-phenylenediame in the presence of a coupling reagent such as EDC//HOBt, followed by treatment with an acid such as acetic acid at elevated temperature to provide the corresponding 5-methoxy-2-(4-benzylpiperidin-1-yl)benzimidazole compound. The latter is converted to compounds of formula I upon treatment with an acid such as HBr at elevated temperature. Compounds of formula I where R2 is OH and R3 is H may be prepared from the corresponding carbonyl compound (i.e. R2+R3=oxo) using a reducing agent such as sodium borohydride.

4-Benzylpiperidin-1-acetic acid compounds may be prepared from a 4-benzylpiperidine derivative and ethyl bromoacetate in the presence of a base such as diisopropylethylamine, followed by hydrolysis of the ester to the corresponding acid. 4-Benzylpiperidine derivatives are known in the art or may be prepared according to conventional organic synthesis procedures. Preparative methods for selected intermediate compounds are provided herein below:

### Preparation of Intermediate A2. 4-(2-fluorobenzyl)piperidine

A neat mixture of 4-fluorobenzyl chloride (10.0 g, 69.0 mmol) and triethylphosphite (11.5 g, 69.0 mmol) was heated to 150° C. and stirred for 15 h. The reaction mixture was concentrated twice from toluene, concentrated and purified by silica gel chromatography (gradient elution: 1:1 hexanes:ethyl acetate (EtOAc) to 10% MeOH/EtOAc) to give the phosphonate.

To a solution of the above phosphonate (16 g, 69 mmol) in 1,3-dimethyl-2-imidazolidinone (25 mL) was added NaH (4.4 g, 110 mmol) followed by slow addition of a solution of N-benzyl-4-piperdinone (13 g, 69 mmol). The reaction mixture was stirred for 20 min, cooled to 0° C., and carefully quenched with water. Dichloromethane (DCM) was added and the layers separated. The organic layer was dried over Na₂SO₄, filtered and concentrated to give 1-benzyl-4-(2-fluorobenzyl)piperidine, which was used without further purification.

To a solution of 1-benzyl-4-(2-fluorobenzyl)piperidine (7 g, 25 mmol) in EtOH (150 mL) was added 10% palladium hydroxide (500 mg). The reaction mixture was plaed on a Parr shaker with 50 psi hydrogen and shaken for 15 h. The reaction mixture was filtered through celite, concentrated and purified by silica gel chromatography (gradient elution, 95:5:0.5 to 90:10:1 dichloromethane: methanol: NH₄OH) to give 4-(2-fluorobenzyl)piperidine: mass spectrum m/z 321 [(M+H)⁺; calcd for C₂₀H₂₄N₄:321].

The procedure used for the preparation of Intermediate A2 was repeated using the appropriate benzyl chloride to provide the following intermediates:

Intermediate A3: 4-(3-Fluorobenzyl)piperidine

Intermediate A4: 4-(4-Fluorobenzyl)piperidine

Intermediate A5: 4-(2,6-Difluorobenzyl)piperidine

Intermediate A6: 4-(4-Methylbenzyl)piperidine

Intermediate A7: 4-(4-Trifluoromethylbenzyl)piperidine

### Preparation of Intermediate A11: trans-4-Benzyl-piperidin-3-ol

### Step 1: Preparation of 1,4-dibenzyl-pyridinium bromide:

A solution of 5 g (29.54 mmol) of 4-benzylpyridine in 25 mL of anhydrous acetone was stirred under N₂, followed by addition of 5.15 g (30.14 mmol, 1.02 equiv.) of benzyl bromide. A yellow suspension formed, followed by a thick white precipitate after 15 min. The suspension was stirred for 18 h., the solid was filtered and washed with ether. The solid was dried under vacuum to yield 9.15 g (91%) of white solid.

### Step 2: Preparation of 1,4-dibenzyl-1,2,3,6-tetrahydropyridine:

The bromide salt, 9.15 g (26.89 mmol), was added to 25 mL each of EtOH and water and cooled to 0° C. Sodium borohydride, 2.04 g (53.78 mmol, 2 equiv.), was added in four equal parts over 15 min., forming an orange suspension. The borohydride was added at a rate keeping the solution<5° C. during addition, then left stir at 0° C. for 2 h., and 18 h. at room temperature. The organics were evaporated in vacuo, followed by addition of 75 mL of water. The aqueous layer was extracted 3×EtOAc, the combined organics were dried over MgSO₄, filtered and evaporated to a pink oil. The oil was dissolved into a minimal amount of CH₂Cl₂ and eluted through a plug of silica gel, flushing through with ˜1 L CH₂Cl₂ then 1 L EtOAc, discarding the first 500 mL containing impurities. The fractions were evaporated to 6.07 g (85.8%) yellow oil.

### Step 3: Preparation of trans-1,4-dibenzyl-piperidin-3-ol:

To a stirring solution of 5.37 g (20.38 mmol) of the alkene in 200 mL of anhydrous tetrahydrofuran (THF) at 0° C. was added 183.49 mL of 1 M borane-THF. The yellow solution was stirred at 0° C. under N₂ for 18 h. The reaction was quenched with 100 mL water. The solution was cooled to 0° C., and 400 mL of 2N NaOH, followed by 50 mL 30% H₂O₂ were added. The mixture was stirred 1 h. at 0° C., then 2.5 h. at reflux. The organics were evaporated in vacuo, and the aqueous reaction mixture was extracted 3×500 mL DCM, collecting 5.7 g (99.3%) of a clear oil.

### Step 4: Preparation of trans-4-benzyl-piperidin-3-ol (A11):

Palladium hydroxide, 1 g, was added to a N₂-purged solution of 5.7 g (20.25 mmol) of the benzylamine in 10 mL EtOH. The mixture was hydrogenated in a Parr Hydrogenation Apparatus @ 55 psi for 36 h. The reaction mixture was filtered through a pad of Celite, and the filtrate was evaporated to 2.9 g (76%) white solid title compound.

### Preparation of Intermediate A12: trans-4-(4-Methylbenzyl)-piperidin-3-ol

Intermediate A12 was prepared using the procedure for A11 by using 4-(4-methylbenzyl)-pyridine (J. Med. Chem. (1990), 33, 3133.) instead of 4-benzylpyridine.

### Preparation of Intermediate C1. (4-benzylpiperidin-1-yl)acetic acid

To a solution of 4-benzylpiperidine (1.0 g, 5.7 mmol) in dimethylformamide (DMF, 20 mL) was added diisopropylethylamine (990 μL, 5.7 mmol), ethylbromoacetate (637 μL, 5.7 mmol), and the reaction mixture was stirred at room temperature for 1 h. The reaction mixture was partitioned between EtOAc and aqueous NaHCO₃, the organic layer was dried over Na₂SO₄, filtered and concentrated. The crude oil was purified by silica gel chromatography (gradient elution, 4:1 hexanes:EtOAc to EtOAc) to give ethyl (4-benzylpiperidin-1-yl)acetate.

Ethyl (4-benzylpiperidin-1-yl)acetate (700 mg, 2.6 mmol) was dissolved in 6N HCl (5 mL) and heated to 100° C. for 1 h. The reaction mixture was cooled and concentrated to give (4-benzylpiperidin-1-yl)acetic acid as a white solid which was used without further purification.

The procedure used for the preparation of Intermediate C1 was repeated using Intermediates A2-A7, A11 and A22 instead of 4-benzylpiperidine to provide the following Intermediates C2-C7, respectively:

Intermediate C2: [4-(2-Fluorobenzyl)piperidin-1-yl]acetic acid

Intermediate C3: [4-(3-Fluorobenzyl)piperidin-1-yl]acetic acid

Intermediate C4: [4-(4-Fluorobenzyl)piperidin-1-yl]acetic acid

Intermediate C5: [4-(2,6-Difluorobenzyl)piperidin-1-yl]acetic acid

Intermediate C6: [4-(4-Methylbenzyl)piperidin-1-yl]acetic acid

Intermediate C7: [4-(4-Trifluoromethylbenzyl)piperidin-1-yl]acetic acid

Intermediate C11: (trans-4-Benzyl-3-hydroxypiperidin-1-yl)acetic acid

Intermediate C12: [trans-4-(4-Methylbenzyl)-3-hydroxypiperidin-1-yl]acetic acid

The following examples are provided to illustrate the present invention are are not to be construed as limiting the scope of the invention in any manner.

### EXAMPLE 1

### 2-[(4-Benzylpiperidin-1-yl)methyl]-1H-benzimidazol-5-ol

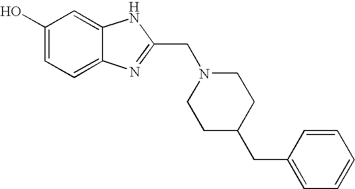

To a solution of (4-benzylpiperidin-1-yl)acetic acid (2.0 g, 7.41 mmol) in DMF (20 mL) was added N-(3-dimethylaminopropyl)-N′-ethylcarbodiimide (EDC, 1.56 g, 8.16 mmol), 1-hydroxy-7-azabenzotriazole (HOAt, 1.11 g, 8.16 mmol), 4-methoxy-1,2-phenylenediamine (1.02 g, 7.41 mmol), and triethylamine (2.06 mL, 14.8 mmol). The reaction mixture was stirred at room temperature for 20 min followed by quenching with aqueous NaHCO₃ and EtOAc. The layers were separated and the organic layer was washed twice with water, dried over Na₂SO₄, filtered and concentrated.

The crude oil was dissolved in acetic acid (20 mL) and heated to 140° C. for 15 min. The reaction mixture was cooled, concentrated twice from toluene and purified by silica gel chromatography and used without further purification. An aliquot of the reaction mixture was purified by silica gel chromatography (gradient elution: 95:5:0.5 to 80:20:2 dichloromethane: methanol: NH₄OH) to give 2-[(4-benzylpiperidin-1-yl)methyl]-5-methoxy-1H-benzimidazole: ¹H NMR (300 MHz, CD₃OD) δ 7.46 (d, 1 H); 7.28 (m, 2 H); 7.20 (t, 1 H); 7.13 (d, 2 H); 7.05 (br.s, 1 H); 6.87 (dd, 1 H); 3.87 (s, 2 H); 3.84 (s, 3 H); 2.94 (d, 2 H); 2.57 (d, 2 H); 2.23 (t, 2 H); 1.68 (d, 2 H); 1.59 (m, 1 H); 1.42 (q, 2 H) ppm; HRMS (ES) m/z 336.2070 [(M+H)⁺; calcd for C₂₁H₂₆N₃O:336.2058].

A solution of 2-[(4-benzylpiperidin-1-yl)methyl]-5-methoxy-1H-benzimidazole was dissolved in HBr/H₂O (48%, 10 mL) was heated to 100° C. for 3 h. The reaction mixture was cooled, concentrated and purified by silica gel chromatography (gradient elution: CH₂Cl₂ to 80:20:2 CH₂Cl₂:MeOH:NH₄OH). The HCl salt was then prepared and triturated in MeOH/Et₂O (1:2) to give the title compound (1.0 g, 38% yield, three steps). HCl salt: ¹H NMR (400 MHz, CD₃OD)δ 7.61 (d, 1 H), 7.24 (t, 2 H), 7.17 (m, 5 H), 4.75 (s, 2 H), 3.60 (d, 2 H), 3.15 (t, 2 H), 2.60 (d, 2 H), 1.91 (m, 3 H); 1.61 (m, 2 H) ppm; HRMS (ES) m/z 322.1914 [(M+H)⁺; calcd for C₂₀H₂₄N₃O:322.1914].

### EXAMPLE 2

### 2-{[4-(2-Fluorobenzyl)piperidin-1-yl]methyl}-1H-benzimidazol-5-ol

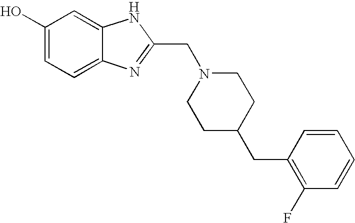

Using the above procedure for Example 1 with [4-(2-fluorobenzyl)-piperidinyl]acetic acid instead of (4-benzylpiperidin-1-yl)acetic acid followed by silica gel chromatography (gradient elution: 95:5:0.5 to 80:20:2 CH₂Cl₂: MeOH: NH₄OH) provided the title compound. The HCl salt was then prepared and recrystallized from iPrOH. HCl salt: ¹H NMR (400 MHz, CD₃OD)δ 7.61 (d, 1 H), 7.23 (m, 2 H), 7.07 (m, 4 H), 4.65 (s, 2 H), 3.59 (d, 2 H), 3.10 (t, 2 H), 2.67 (d, 2 H), 1.93 (d, 3 H), 1.62 (m, 2 H) ppm; HRMS (ES) m/z 340.1821 [(M+H)⁺; calcd for C₂₀H₂₃FN₃O:340.1820].

### EXAMPLE 3

### 2-{[4-(3-Fluorobenzyl)piperidin-1-yl]methyl})-1H-benzimidazol-5-ol

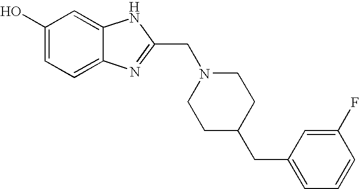

Using the above procedure for Example 1 with [4-(3-fluorobenzyl)-piperidin-1-yl]acetic acid instead of (4-benzylpiperidin-1-yl)acetic acid followed by silica gel chromatography (gradient elution: 95:5:0.5 to 80:20:2 CH₂Cl₂:MeOH:NH₄OH) provided the title compound. The HCl salt was then prepared and recrystallized from iPrOH. HCl salt: ¹H NMR (300 MHz, CD₃OD) δ 7.70 (d, 1 H), 7.30 (m, 1 H), 7.17 (m, 2 H), 6.93 (m, 3 H), 4.90 (s, 2 H), 3.63 (d, 2 H), 3.22 (t, 2 H), 2.62 (d, 2 H), 1.92 (d, 3 H), 1.63 (m, 2 H) ppm; HRMS (ES) m/z 340.1821 [(M+H)⁺; calcd for C₂₀H₂₃FN₃O:340.1820].

### EXAMPLE 4

### 2-{[4-(4-Fluorobenzyl)piperidin-1-yl]methyl}-1H-benzimidazol-5-ol

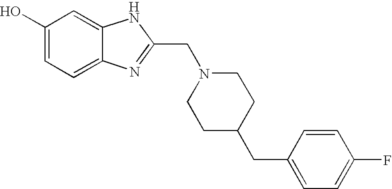

Using the above procedure for Example 1 with [4-(4-fluorobenzyl)-piperidin-1-yl]acetic acid instead of (4-benzylpiperidin-1-yl)acetic acid followed by silica gel chromatography (90:10:1 CH₂Cl₂:MeOH:NH₄OH) provided the title compound. Free base: ¹H NMR (400 MHz, CDCl₃) δ 7.40 (br s, 1 H); 7.28 (s, 1 H); 7.05 (m, 2 H); 6.92 (m, 2 H); 6.81 (d, 1 H); 3.75 (s, 2 H); 2.91 (d, 2 H); 2.47 (d, 2 H); 2.12 (t, 2 H); 1.58 (m, 2 H); 1.50 (m, 1 H); 1.28 (m, 2 H) ppm; HRMS (ES) m/z 340.1808 [(M+H)⁺; calcd for C₂₀H₂₃FN₃O:340.1820].

### EXAMPLE 5

### 2-{[4-(2,6-Difluorobenzyl)piperidin-1-yl]methyl}-1H-benzimidazol-5-ol

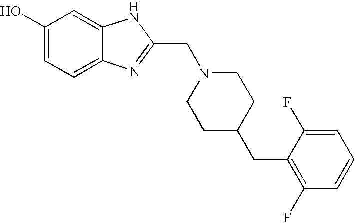

Using the above procedure for Example 1 with [4-(2,6-difluorobenzyl)-piperidin-1-yl]acetic acid instead of (4-benzylpiperidin-1-yl)acetic acid followed by silica gel chromatography (gradient elution: 95:5:0.5 to 80:20:2 CH₂Cl₂: MeOH: NH₄OH) provided the title compound. The HCl salt was then prepared and recrystallized from iPrOH. HCl salt: ¹H NMR (300 MFz, CD₃OD) δ 7.65 (d, 1 H), 7.29 (m, 1 H), 7.15 (m, 2 H), 6.95 (t, 2 H), 4.72 (s, 2 H), 3.61 (d, 2 H), 3.14 (t, 2 H), 2.72 (d, 2 H), 1.92 (d, 3 H), 1.70 (m, 2 H) ppm; HRMS (ES) m/z 358.1725 [(M+H)⁺; calcd for C₂₀H₂₂F₂N₃O: 358.1726].

### EXAMPLE 6

### 2-{[4-(4-Methylbenzyl)piperidin-1-yl]methyl}-1H-benzimidazol-5-ol

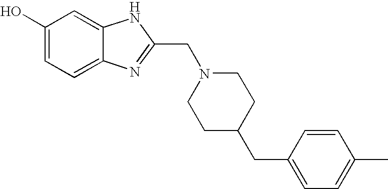

Using the above procedure for Example 1 with [4-(4-methylbenzyl)-piperidinyl]acetic acid instead of (4-benzylpiperidin-1-yl)acetic acid followed by silica gel chromatography (gradient elution: 90:10:1 to 80:20:2 CH₂Cl₂: MeOH: NH₄OH) provided the title compound. Free base: ¹H NMR (400 MHz, CDCl₃) δ 7.39 (br.s, 1H); 7.28 (s, 1H); 7.10 (d, 2H); 6.95 (d, 2H); 6.81 (d, 1H); 3.70 (s, 2H); 2.90 (d, 2H); 2.40 (d, 2H); 2.29 (s, 3H); 2.10 (t, 2H); 1.57 (m, 2H); 1.48 (m, 1H); 1.23 (m, 2H) ppm; HRMS (ES) m/z 336.2082 [(M+H)⁺; calcd for C₂₁H₂₆N₃O:336.2071].

### EXAMPLE 7

### 2-{[4-(4-Trifluoromethylbenzyl)piperidin-1-yl]methyl}-1H-benzimidazol-5-ol

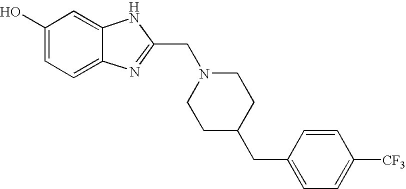

Using the above procedure for Example 1 with [4-(4-trifluoromethylbenzyl)-piperidinyl]acetic acid instead of (4-benzylpiperidin-1-yl)acetic acid followed by silica gel chromatography (gradient elution: 90:10:1 to 80:20:2 CH₂Cl₂: MeOH: NH₄OH) provided the title compound: mass spectrum m/z 390 [(M+H)⁺; calcd for C₂₁H₂₃F₃N₃O:390].

### EXAMPLE 8

### 2-[(4-Benzyl-4-hydroxypiperidin-1-yl)methyl]-1H-benzimidazol-5-ol

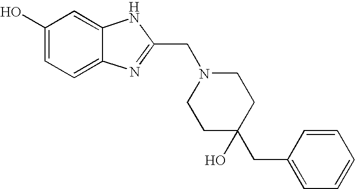

The title compound was prepared by following the above procedure for Example 1 except using 4-benzyl-piperidin-4-ol instead of 4-benzylpiperidine: mass spectrum m/z 338 [(M+H)⁺; calcd for C₂₀H₂₄N₃O₂:338].

### EXAMPLE 9

### {1-[(5-hydroxy-1H-benzimidazol-2-yl)methyl]piperidin-4-yl}(phenyl)methanone

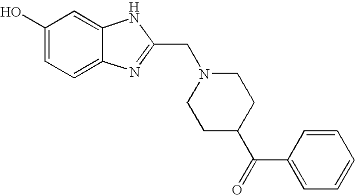

The title compound was prepared by following the above procedure for Example 1 except using phenyl(piperidin-4-yl)methanone instead of 4-benzylpiperidine: mass spectrum m/z 336[(M+H)⁺; calcd for C₂₀H₂₂N₃O₂:336].

### EXAMPLE 10

### 2-({4-[Hydroxy(phenyl)methyl]piperidin-1-yl}methyl)-1H-benzimidazol-5-ol

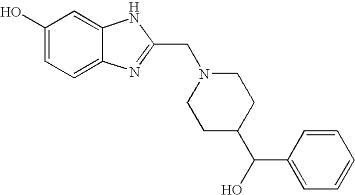

To a solution of the compound of Example 9 (50 mg, 0.15 mmol) in MeOH (5 mL) was added sodium borohydride (11 mg, 0.30 mmol) at rt. The reaction mixture was stirred for 5 min, quenched with H₂O and extracted with EtOAc (2×). The combined organic layers were dried over Na₂SO₄, filtered, concentrated, and purified by silica gel chromatography (gradient elution 95:5:0.5 to 90:10:1 CH₂Cl₂: MeOH:NH₄OH). The HCl salt was then prepared and triturated in MeOH/Et₂O (1:2) to give the title compound: ¹H NMR (300 MHz, CD₃OD) δ 7.70 (d, 1 H), 7.30 (m, 5 H), 7.18 (m, 2 H), 4.80 (s, 2 H), 4.40 (d, 1 H), 3.65 (dd, 2 H), 3.21 (dd, 2 H), 2.20 (d, 1 H), 2.00-1.60 (m, 4 H) ppm; HRMS (ES) m/z 338 [(M+H)⁺; calcd for C₂₀H₂₄N₃O₂:338].

### EXAMPLE 11

### 2-[(trans-4-Benzyl-3-hydroxypiperidin-1-yl)methyl]-1H-benzimidazol-5-ol

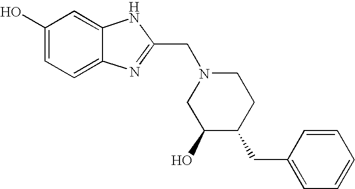

Using the above procedure for Example 1 with (trans-4-benzyl-3-hydroxypiperidin-1-yl)acetic acid instead of (4-benzylpiperidin-1-yl)acetic acid, followed by silica gel chromatography (gradient elution: 95:5:0.5 to 80:20:2 CH₂Cl₂: MeOH:NH₄OH) provided the title compound. The HCl salt was then prepared and recrystallized from iPrOH. HCl salt: ¹H NMR (400 MHz, CD₃OD) δ 7.64 (d, 1 H), 7.27 (m, 2 H), 7.18 (m 5 H), 4.68 (s, 2 H), 3.70 (m, 1 H), 3.55 (d, 1 H), 3.40 (d, 1 H), 3.20 (d, 1 H), 2.91 (m, 2 H), 2.42 (dd, 1 H), 1.83 (m, 2 H), 1.56 (m, 1 H) ppm; HRMS (ES) m/z 338.1862 [(M+H)⁺; calcd for C₂₀H₂₄N₃O₂: 338.1863].

### EXAMPLE 12

### 2-{[trans-3-hydroxy-4-(4-methylbenzyl)piperidin-1-yl]methyl}-1H-benzimidazol-5-ol

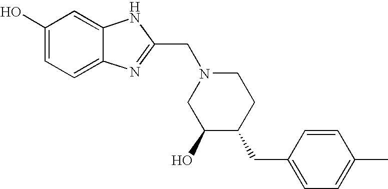

Using the above procedure for Example 1 with [trans-4-(4-methylbenzyl)-3-hydroxypiperidin-1-yl]acetic acid instead of (4-benzylpiperidin-1-yl)acetic acid followed by silica gel chromatography (gradient elution: 95:5:0.5 to 80:20:2 CH₂Cl₂:MeOH:NH₄OH) provided the title compound. The HCl salt was then prepared and recrystallized from iPrOH. HCl salt: ¹H NMR (400 MHz, CD₃OD) δ 7.61 (d, 1 H), 7.08 (m, 6 H), 4.50 (s, 2 H), 3.62 (m, 1 H), 3.41 (m, 1 H), 3.23 (m, 1 H), 3.16 (d, 1 H), 2.77 (m, 2 H), 2.39 (dd, 1 H), 2.29 (s, 3 H), 1.83 (m, 1 H), 1.71 (m, 1 H), 1.48 (m, 1 H) ppm; HRMS (ES) m/z 352.2022 [(M+H)⁺; calcd for C₂₁H₂₅N₃O₂:352.2020].

What is claimed is:

1. A compound having the formula I: 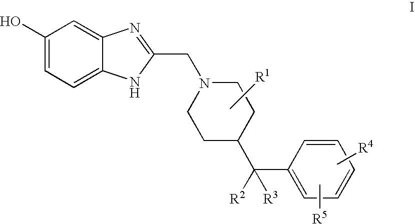
and pharmaceutically acceptable salts thereof, wherein
R¹ is H or OH;
R² is H or OH;
R³ is H, or R³ and R² together represent oxo;
R⁴ and R⁵ are independently H, halogen, C₁₋₆alkyl, C₁₋₆alkoxy or trifluoromethyl.

2. A compound of claim 1 wherein R¹, R² and R³ are each H.

3. A compound of claim 1 wherein R¹ is OH, and R² and R³ are each H.

4. A compound of claim 1 wherein R² and R³ together represent oxo.

5. A compound selected from

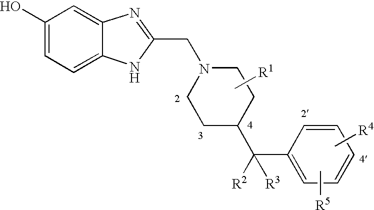

| R¹ | R² | R³ | R⁴ | R⁵ |
|---|---|---|---|---|
| H | H | H | H | H |
| H | H | H | H | 2′-F |
| H | H | H | H | 3′-F |
| H | H | H | H | 4′-F |
| H | H | H | 2′-F | 6′-F |
| H | H | H | H | 4-CH₃ |
| H | H | H | H | 4-CF₃ |
| 4-OH | H | H | H | H |
| H | oxo | | H | H |
| H | OH | H | H | H |
| 3-OH | H | H | H | H |
| 3-OH | H | H | H | 4-CH₃ |

or a pharmaceutically acceptable salt thereof.

6. A pharmaceutical composition comprising an inert carrier and an therapeutically effective amount of a compound according to claim 1.

7. A method of treating pain comprising a step of administering to one in need of such treatment a therapeutically effective amount of a compound according to claim 1.

8. The method of claim 7 wherein said pain is neuropathic pain.

9. A method of treating migraine, depression, anxiety, schizophrenia, or stroke.

10. A method of treating Parkinson's disease comprising a step of administering to one in need of such treatment a therapeutically effective amount of a compound according to claim 1.
